(19) United States
(12) Patent Application Publication
(10) Pub. No.: US 2024/0091135 A1
(43) Pub. Date: Mar. 21, 2024

(54) COMPOSITIONS AND METHODS FOR PROMOTING ORAL HEALTH

(71) Applicant: Reshma N. Kheraj, Wake Forest, NC (US)

(72) Inventor: Reshma N. Kheraj, Wake Forest, NC (US)

(21) Appl. No.: 18/095,793

(22) Filed: Jan. 11, 2023

Related U.S. Application Data
(60) Provisional application No. 63/407,631, filed on Sep. 17, 2022.

(51) Int. Cl. A61K 8/99; A61K 8/60; A61Q 11/00

(52) U.S. Cl. CPC A61K 8/99; A61K 8/60; A61Q 11/00

(57) ABSTRACT

A composition and method for improving oral health by restoring, maintaining, and/or promoting natural flora and fauna of the oral cavity. The composition includes one or more probiotic and/or prebiotic strains. The composition is applied to the oral cavity.

### CROSS-REFERENCE TO RELATED APPLICATIONS

This application claims priority from the U.S. provisional patent application Ser. No. 63/407,631, filed on Sep. 17, 2022, which is incorporated herein by reference in its entirety.

### FIELD OF INVENTION

The present invention relates to a composition for the oral cavity, and more particularly, the present invention relates to a composition having Prebiotics and/or Probiotics for the oral cavity.

### BACKGROUND

Oral health is of utmost importance like any other part of the body. Oral health is important for the overall well-being of an individual. Oral health may also affect the personality of a person. Several studies have linked oral health with a number of diseases, such as heart disease. A number of products are available for maintaining oral health of which dentifrices are the most common. The dentifrices are used with toothbrushes for cleaning the teeth. Mouthwashes are also commonly used that include odor-inhibiting substances and optionally antimicrobials. However, the available products are insufficient for maintaining proper health.

A need is therefore appreciated for a novel composition that promotes oral health and overall well-being.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a composition for oral health that promotes healthy flora and fauna in the oral cavity and gut.

It is another object of the present invention that the composition promotes overall well-being.

It is still another object of the present invention that the composition provides for maintaining a healthy heart.

It is yet an object of the present invention that the composition can be implemented in a variety of forms and products.

In one aspect, disclosed is a novel composition for the oral cavity and a method of use therefor in restoring, maintaining, and/or promoting oral health.

In one aspect, disclosed is a method for improving oral health, the method comprises providing a composition comprising one or more probiotic and/or prebiotic strains; and applying the composition into an oral cavity, wherein oral mucosa of the oral cavity is exposed to the composition for a pre-determined duration. The one or more probiotic strains are selected from a group consisting of Bifidobacterium animalis, Bifidobacterium lactis, Bifidobacterium longum, Bifidobacterium bifidum, Lactobacillus acidophilus, Lactobacillus bulgaricus, Lactobacillus casei, Lactobacillus gasseri, Lactobacillus plantarum, Lactobacillus rhamnosus, Lactobacillus salivarius, Lactococcus lactis Subsp. Lactis, Lactococcus lactis Subs. Lactis biovardiacetylactis, Leuconostoc pseudomesenteroides, Streptococcus thermophilus, and a combination thereof. The composition further comprises one or more prebiotics selected from a group consisting of Fructo-oligosaccharides, galacto-oligosaccharides, trans-galacto-oligosaccharides, and a combination thereof. The composition is in the form of a toothpaste, the toothpaste is applied to teeth. The composition is in the form of a mouthwash. The composition is the form of chewing gum. The composition is the form of a spray formulation, and the composition is applied by spraying in the oral cavity. The composition is the form of frozen formulation, wherein the composition is applied as the frozen formulation.

### DETAILED DESCRIPTION

Subject matter will now be described more fully hereinafter. Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is a composition of the oral cavity that helps to restore, maintain, or promote the flora and fauna of oral activity and the gut. The disclosed composition can include one or more probiotics, and optionally also includes prebiotics. The disclosed composition by maintaining the essential flora and fauna of the oral cavity helps to maintain oral health. The disclosed composition includes one or more probiotics, wherein the choice of probiotics may depend upon several factors, such as synergy between the probiotic strains, occupation of a person, phenotype of the person, and the like factors. For example, depending on the occupation, a choice of probiotic strains can be made. Athletes may have a different set of requirements than a person suffering from bad breath. Moreover, the choice of probiotic strains may also depend upon the formulation.

In one implementation, the probiotic strains can be selected from a group including Bifidobacterium animalis, Bifidobacterium lactis, Bifidobacterium longum, Bifidobacterium bifidum, Lactobacillus acidophilus, Lactobacillus bulgaricus, Lactobacillus casei, Lactobacillus gasseri, Lactobacillus plantarum, Lactobacillus rhamnosus, Lactobacillus salivarius, Lactococcus lactis Sub Sp. Lactis, Lactococcus lactis Subs. Lactis biovardiacetylactis, Leuconostoc pseudomesenteroides, Streptococcus thermophilus, or in combinations thereof.

In one implementation, the disclosed composition can also include suitable prebiotics, such as Fructo-oligosaccharides (FOS), galacto-oligosaccharides (GOS), and trans-galacto-oligosaccharides (TOS). Prebiotics are beneficial in promoting the growth of healthy bacteria.

In one implementation, the disclosed composition can also include analgesic, and more specifically topical analgesics, such as lidocaine, benzocaine, and menthol. The topical analgesics can help decrease pain and inflammation in the gums, while the disclosed composition may aid in healing the gums and oral cavity.

In one implementation, the disclosed composition can include additional ingredients to promote well-being. Examples of such ingredients include Purple Coneflower, Grape Seed Extract, Hyaluronic Acid, Folic Acid, Collagen Type One, and Three, Magnesium Citrate, Vitamin D3, Vitamin C, Vitamin K, Calcium, Iron, Lemongrass, Baking soda, Charcoal, and Green Tea.

The disclosed composition can be formulated in a variety of formulations including liquid, solid, paste, serum, gel, emulsion, and powder form. The disclosed composition can include additional ingredients which may be functional and neutral. The functional ingredients may have a health benefit, such as abrasives can be added for cleaning the teeth. Neutral ingredients may be needed for a formulation, and the use of such neutral ingredients in a formulation is well-known in the art.

In one implementation, the disclosed composition including the probiotic strains and one or more prebiotics can be formulated as toothpaste. The composition can include abrasives for cleaning the teeth, and the use of such abrasives is known to a skilled person and any such abrasive known for use in toothpaste is within the scope of the present invention. Examples of such abrasives include aluminum hydroxide, calcium carbonate, magnesium carbonate, sodium bicarbonate, calcium hydrogen phosphates, silicas and zeolites, and hydroxyapatite. Similarly, fragrances, flavors, coloring agents, and the like can also be incorporated. Sodium fluoride can also be added for preventing tooth decay and gum diseases. For sensitive gums, ingredients such as potassium nitrate that can soothe the nerves and stannous fluoride to protect dentin can also be incorporated. Using the disclosed composition as toothpaste has the advantage that the oral cavity/mucosa is exposed to the disclosed composition for a longer duration enhancing the effectiveness of the disclosed composition in promoting flora and fauna of the oral cavity, and thus improving oral health. The toothpaste can be in the form of a semi-solid paste, foam paste, or toothpaste gel.

The disclosed composition can also be formulated in the form of mouthwash. The mouthwash composition can also include a solvent, antimicrobial, essential oils, and/or odor-inhibiting agents.

The disclosed composition can also be formulated as a spray. In spray form, the disclosed composition can be easily and quickly applied to the oral mucosa. Moreover, when the frequent application of disclosed composition may be prescribed, the spray formulation can be particularly useful. The spray formulation can include solvents, flavors, and the like.

In a preferred embodiment, the disclosed composition can also be formulated as chewing gum. The chewing gums can provide for the slow and longer release of probiotics in the oral cavity. The chewing gum can include ingredients known to a skilled person for use in making chewing gum. The chewing gums also include gel-filled gums. The disclosed composition can also be formulated as Gummies, candies, and the like.

The disclosed composition can also be formulated in a sustained release form using topical patches that may be applied to oral mucosa for slow and sustained release of probiotics in the oral cavity. Creams containing the disclosed composition can be applied to the oral mucosa.

In one implementation, the disclosed composition can also be formulated in a frozen form. The disclosed composition can be administered to the oral cavity in the frozen form. Such a frozen form of the disclosed composition can be useful in certain inflammations and swellings.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for improving oral health, the method comprises:
providing a composition comprising one or more probiotic strains; and
applying the composition into an oral cavity, wherein oral mucosa of the oral cavity is exposed to the composition for a pre-determined duration.

2. The method according to claim 1, wherein the one or more probiotic strains are selected from a group consisting of Bifidobacterium animalis, Bifidobacterium lactis, Bifidobacterium longum, Bifidobacterium bifidum, Lactobacillus acidophilus, Lactobacillus bulgaricus, Lactobacillus casei, Lactobacillus gasseri, Lactobacillus plantarum, Lactobacillus rhamnosus, Lactobacillus salivarius, Lactococcus lactis Subsp. Lactis, Lactococcus lactis Subs. Lactis biovardiacetylactis, Leuconostoc pseudomesenteroides, Streptococcus thermophilus, and a combination thereof.

3. The method according to claim 1, wherein the composition further comprises one or more prebiotics selected from a group consisting of Fructo-oligosaccharides, galacto-oligosaccharides, trans-galacto-oligosaccharides, and a combination thereof.

4. The method according to claim 1, wherein the composition is in a form of a toothpaste, the toothpaste is applied to teeth.

5. The method according to claim 1, wherein the composition is in a form of a mouthwash.

6. The method according to claim 1, wherein the composition is a form of chewing gum.

7. The method according to claim 1, wherein the composition is a form of a spray formulation, and the composition is applied by spraying in the oral cavity.

8. The method according to claim 1, wherein the composition is a form of frozen formulation, wherein the composition is applied as the frozen formulation.
